(19) United States
(12) Patent Application Publication
(10) Pub. No.: US 2020/0253862 A1
(43) Pub. Date: Aug. 13, 2020

(54) THERMALLY NEUTRAL INHALATION GAS COMPOSITION

(71) Applicant: MONATOMICS TECHNOLOGY, Paris (FR)

(72) Inventor: Hélène DAVID, Saint-Laurent de l'lle d'Orleans (CA)

(21) Appl. No.: 16/863,836

(22) Filed: Apr. 30, 2020

Related U.S. Application Data
(62) Division of application No. 16/091,147, filed on Oct. 4, 2018.

(51) Int. Cl. A61K 9/00; A61K 33/00

(52) U.S. Cl. CPC A61K 9/007; A61K 33/00

(57) ABSTRACT

The present application relates to methods to administer to a patient a thermally neutral inhalation gas composition that includes oxygen and a mixture of inert gases. The mixture of inert gases includes a first compound such as xenon, and a second compound having hypothermal properties, such as helium. In the methods, a thermally neutral inhalation gas composition including oxygen and a mixture of inert gases is selected and the thermally neutral inhalation gas composition is administered to the patient at an inhalation temperature of a specified range such that the body temperature of the patient is maintained at a specified temperature.

The present invention relates to an inhalation gas composition and, more specifically, choosing appropriate proportions of gas for the composition.

Within the framework of an ischemia-reperfusion and, as an example in the case of a stroke, neonatal encephalopathy, or a treatment-related ischemia, such as ischemia due to an organ transplant or a clamp placement during a surgical operation, particularly clamp surgery. Traditionally, controlled hypothermia is induced in order to protect the brain and reduce cellular metabolism.

Xenon is an anesthetic agent that has been authorized on the European market since 2007. The reason xenon has organo-protective, particularly neuro-protective, properties is probably due to its being an antagonist of N-methyl-D-aspartate (NMDA) glutamate receptors and due to its anti-proteolytic effect. (“Xenon: elemental anesthesia in clinical practice,” Robert D. Sanders, Daqing Ma and Mervyn Maze, British Medical Bulletin (2005) 71 (1): 115-135).

Studies have also shown that argon, the agonist of type A GABAergic receptors (“Gamma-aminobutyric acid neuropharmacological investigations on narcosis produced by nitrogen, argon, or nitrous oxide,” Abraini J H, Kriem B, Balon N, Rostain J C, Risso J J, Anesthesia and Analgesia 2003; 96:746-9) and antagonists of Mu-type opioidergic receptors (“Argon blocks the expression of locomotor sensitization to amphetamine through antagonism at the vesicular monoamine transporter-2 and mu-opioid receptor in the nucleus accumbens,” David H N, Dhilly M, Degoulet M, Poisnel G, Meckler C, Vallée N, Blatteau J É, Risso J J, Lemaire M, Debruyne D, Abraini J H, Translational Psychiatry 2015; 5:e594) has organo-protective, particularly neuro-protective properties (“Argon: Systematic Review on Neuro- and Organo-protective Properties of an “Inert” Gas,” A. Höllig, A. Schug, A V. Fahlenkamp, R. Rossaint, M. Coburn and Argon Organo-Protective Network (AON), International Journal of Molecular Sciences. 2014 October; 15(10): 18175-18196)).

In any case, xenon and argon have the disadvantage of having hyperthermic properties for the given inhalation temperatures, since these inert gases have a higher molar mass than nitrogen and a lower thermal conductivity than nitrogen, which gives them, when used in inhalation gases, a hyperthermic character. However, using a gas with hyperthermic properties tends to induce hyperthermia in the subjects breathing it, which is detrimental to the treatment of most neurological or psychiatric diseases.

In this context, the purpose of the invention is an inhalation gas composition that includes oxygen as well as a mixture of inert gases. The mixture of inert gases includes a first compound chosen from xenon and argon, showing hyperthermic properties, and a second compound with hypothermic properties, the said gas mixture including proportions of the first compound and the second compound as the said mixture of inert gases is hypothermic in pre-determined temperature conditions.

By an “inhalation” gas composition, we mean a gas composition with at least 21% oxygen, so that it can be breathed by the subject, since if the inhaled mixture contains less than 21% oxygen, the subject will go into hypoxia.

Reflecting what was defined above, we understand that a gas with hypothermic properties is defined as a gas or mixture with a lower molar mass than nitrogen, and a higher thermal conductivity than nitrogen, making it possible to put the subject breathing the gas in a state of hypothermia.

Thus, a “thermically neutral” mixture is defined as a mixture with substantially the same thermal properties as atmospheric nitrogen at a given temperature, meaning, in other words, that the gas composition inhaled at a given temperature makes it possible to maintain the body temperature of the subject inhaling the gas within a normal temperature, from 36° C. to 38° C.

We understand that the inhalation of such a composition for inhalation temperatures between 16° C. and 28° C. makes it possible to maintain bodily hypothermia, meaning keeping the body temperature within a hypothermic range, which is a temperature range below the body's normal variability, substantially between 36.1° C. and 37.8° C. (Simmers, Louise. Diversified Health Occupations. 2nd ed. Canada: Delmar, 1988: 150-151). This range can be rounded to 36-38° C., or 37±1° C. In other words, the invention makes it possible to supply a gas composition that does not increase or risk increasing the body temperature of the subjects inhaling the composition outside of a value range considered normal, which is between 36° C. and 38° C.

According to a feature of the invention, the second compound with hypothermic properties also shows organo-protective properties. By organo-protective properties, we mean the protection of internal organs, such as the brain, blood vessels and nerves. Thus, on top of maintaining the body temperature within a value range that corresponds to a therapeutic body hypothermia, according to the invention, the inhalation gas composition makes it possible to protect the internal organs during the subject's inhalation.

More specifically, the second compound can more efficiently be helium. Indeed, helium has more hypothermic and organo-protective properties. (“Heliox and oxygen reduce infarct volume in a rat model of focal ischemia,” Pan Y, Zhang H, Van Deripe D R, Cruz-Flores S, Panneton W M (2007), Experimental Neurology 205:587-90; “The effect of helium-oxygen mixtures on body temperature,” Tapper D, Arensman R, Johnson C, Folkman J (1974), Journal of Pediatric Surgery 9:597-603; “Post-ischemic helium provides neuroprotection in rats subjected to middle cerebral artery occlusion-induced ischemia by producing hypothermia,” David H N, Haelewyn B, Chazalviel L, Lecocq M, Degoulet M, Risso J J, Abraini J H (2009), Journal of Cerebral Blood Flow & Metabolism 29:1159-1165; “Modulation by the Noble Gas Helium of Tissue Plasminogen Activator: Effects in a Rat Model of Thromboembolic Stroke,” Haelewyn B, David H N, Blatteau J E, Vallee N, Meckler C, Risso J J, Abraini J H (2016), Critical Care Medicine in press).

The inhalation gas composition comprises 50% to 79% of the mixture of inert gases; these proportions make it possible to ensure that the composition can be inhaled and to prevent hypoxia in the subject inhaling the composition.

According to a first set of characteristics of the invention, taken alone or in combination, in the context of applying a first compound in the form of xenon, we can expect that:
the said composition contains at between 7 to 50% xenon. Limiting the xenon content to below 50% prevents an anesthetic effect on the subject breathing the composition, also limiting the cost of obtaining the composition.
the said composition contains at most 71% helium.

According to one of the invention's modes of operation, for inhalation temperatures higher or equal to 23° C., we can expect the said composition to include 21 to 30 oxygen, 11 to 64% helium, and 13 to 45% xenon. More specifically, for a 22% oxygen rate, the composition can have 42 to 49% helium and 29 to 36% xenon or 25% oxygen, 40 to 48% helium and 27 to 35% xenon, in order to ensure a body temperature between 36 and 38° C. in humans. For example, to get a 37° C. body temperature, the said composition can substantially have 22% oxygen, 43% helium and 35% xenon. By substantially, we mean that a 1% margin of error or uncertainty is admissible.

According to a first set of characteristics of the invention, taken alone or in combination, in the context of applying a first compound in the form of argon, we can expect that:
the said composition has at least 11% argon.
the said composition has at least 67% helium.

According to one of the invention's modes of operation, we can expect the said composition to include 21% to 25% of oxygen, 3% to 28% of helium, and 49% to 76% of argon. More specifically, when the composition is inhaled at a temperature of 22° C., it can include 22% of oxygen, 7% to 22% of helium and 56% to 71% of argon, or the said composition can include 25% of oxygen, 7% to 21% of helium and 54% to 68% of argon, in order to guarantee a body temperature of between 36° C. and 37° C. in humans.

Other characteristics, details and advantages of the invention will be clarified in the description below, for informational proposes with regard to the drawings in which:

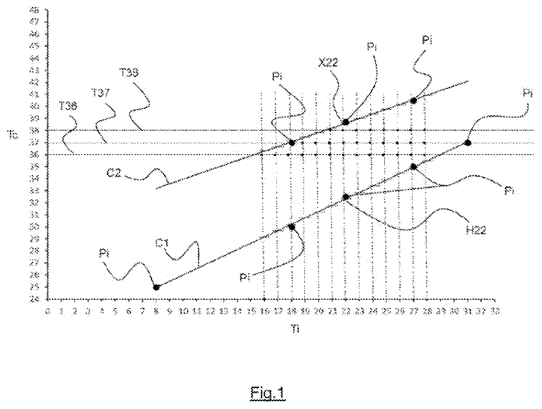

FIG. 1 is a graphic representation of a rat's body temperature based on the temperature of the gas inhaled, which is helium (curve C1) or xenon (curve C2);

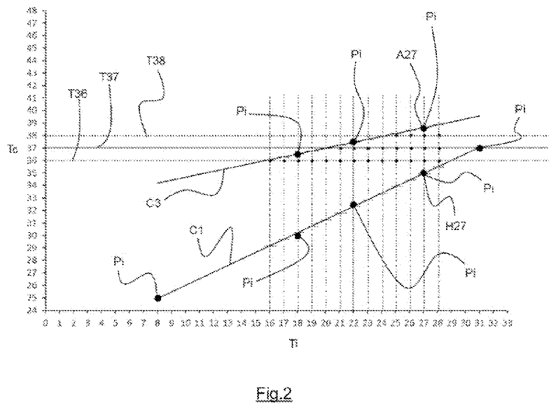

FIG. 2 is a graphic representation of a rat's body temperature based on the temperature of the gas inhaled, which is helium (curve C1) or argon (curve C3);

table 1 in the annex represents the physical properties of the compounds of the present invention;

table 2 in the annex represents the proportions of xenon and helium based on the proportion of oxygen, the inhalation temperature of the composition and its effect on body temperature measured in a rat;

table 3 in the annex represents the proportions of xenon and helium based on the proportion of oxygen, the inhalation temperature of the composition and its effect on body temperature measured in a rat.

Air is mainly composed of 21% oxygen, 78% nitrogen and 1% noble gas. It is substantially equivalent to say that the reference air is composed of 21% oxygen and 79% nitrogen, since this oxygen content is the minimum value that a gaseous mixture must contain to prevent hypoxia of a subject inhaling such a gaseous mixture. According to the invention, the gaseous composition includes oxygen and a mixture of inert gases, since the proportion of nitrogen in the air is replaced with the mixture of inert gases.

This mixture of inert gases is composed of a first compound with hyperthermic properties and a second compound with hypothermic properties. The proportions of each inert gas mixture composition are those that allow the gaseous composition inhaled to maintain a subject's body temperature within a hypothermic temperature range going from 36° C. to 38° C.

The composition contains at least 21% oxygen in order to prevent any hypoxia during inhalation. The composition contains at least 50% oxygen and preferably between 21% and 30%, or even 21% and 25%. Thus, the composition contains at least 50% of the inert gas mixture, but preferably from 70% to 79%.

The inert gas mixture contains a first compound chosen from inert gases with hyperthermic properties and a second compound chosen from inert gases with hypothermic properties. The inert gases have the advantage of not being metabolized after being inhaled.

The first compound chosen from inert gases with hyperthermic properties is xenon or argon. Indeed, as shown in table 1 in the annex, xenon and argon have a higher molar mass than nitrogen and a lower thermal conductivity than nitrogen, which gives them a hyperthermic character when one or the other replaces nitrogen in a gaseous mixture.

In addition to having hyperthermic properties, xenon and argon have organo-protective properties, meaning that these compounds help protect organs, blood vessels and nerves. These compounds are likely to protect the brain.

Below we describe a first method for operating the invention, in which the gaseous composition includes as a first compound, meaning as a compound with hyperthermic properties, xenon.

Xenon is mixed with a gas with hypothermic properties in proportions that make the mixture have hypothermic properties. In the following, we choose an inert gas to be mixed with xenon. We specifically choose a gas with hypothermic properties, namely helium. Indeed, as shown in table 1 in the annex, helium has a lower molar mass than nitrogen and a higher thermal conductivity than nitrogen, which gives it a hypothermic character when it replaces nitrogen in a gaseous mixture. On the other hand, helium also has organo-protective properties.

In order to offer a hypothermic gaseous composition, meaning one that does not change the body temperature of the subjects inhaling the composition outside of a temperature range between 36° C. and 38° C., the proportions of the first and second composite mixture of inert gases must be precisely calculated. These proportions are extrapolated from experimental data retrieved with the gases composing the mixture. These experimental data, obtained from a rat whose body temperature was deemed normal, are similar to the normal human body temperature, ranging from 35.9° C. and 37.5° C. (Animal care and use committee, Johns Hopkins University, http://web.jhu.edu/animalcare/procedures/rat.html), and were used to make the graphs in FIGS. 1 and 2.

The graph of FIG. 1, which represents the experimental data of the body temperature Tc taken from a rat based on the inhalation temperature Ti of a helium-oxygen mixture (curve C1) or a xenon-oxygen mixture (curve C2), helps determine the proportions of the gaseous composition to respect to get a hypothermic gaseous mixture based on the inhalation temperature. To go into greater detail, curves C1 and C2 correspond to regression lines obtained based on the said experimental data Pi, examples of which were shown in FIG. 1.

Experimental data were obtained as follows: Rats were placed for 3 hours in a closed chamber pumped with a continuous flow of a gaseous mixture, containing 22% oxygen (O₂) and 78% helium, xenon or argon (He, Xe or Ar). This gaseous mixture was administered at different temperatures. The gaseous mixture flowed at 10 L/min and kept the concentration of carbon dioxide (CO₂) below 0.03% and humidity around 60% and 70%. The gas mixtures were obtained using mass flow meters of an absolute precision of 0.2% of the value displayed (e.g. displayed value 78%, precision=0.16%, or 78+/−0.16%); the oxygen concentration was controlled using a specific analyzer. After 3 hours of exposure, for each administration temperature, the rats' rectal body temperature was measured.

The rat is commonly used as a pre-clinical model for studying the physiology and pathologies of humans, since the normal body temperatures Tc of rats and humans are similar. Administering a gaseous mixture of different temperatures in the rat in an enclosed space can be compared to administering this kind of gas mixture to a human, where the inhalation temperature Ti is substantially equal to the room temperature where the gaseous treatment is administered. The inhalation temperature Ti can, for example, run from 16° C. to 28° C.

For an inhalation temperature of 22° C., we determine:
H22 and X22 points, respectively located on the helium C1 and xenon C2 curves.
horizontal lines T36, T37 and T38 correspond to target body temperatures of 36° C., 37° C., and 38° C.

By doing so, for a distance of H22-X22, a distance that represents the sum of the percentages of helium and xenon in the inhalation gas composition containing oxygen, xenon and helium, we get:
A distance X22-T36, which represents the proportion of helium that maintains body temperature Tc at 36° C.,
A distance H22-T36, which represents the proportion of xenon that maintains body temperature Tc at 36° C.,
A distance X22-T37, which represents the proportion of helium that maintains body temperature Tc at 36° C.,
A distance H22-T37, which represents the proportion of xenon that maintains body temperature Tc at 37° C.,
A distance X22-T38, which represents the proportion of helium that maintains body temperature Tc at 38° C.,
A distance H22-T38, which represents the proportion of xenon that maintains body temperature Tc at 38° C.,

Using these experimental data, we created table 2 in the annex, which shows the proportions of a mixture between helium and xenon, bearing in mind the proportion of oxygen. It is clear that these proportions of helium and xenon depend both on the temperature of the inhaled gas Ti, the proportion of oxygen present in the gaseous composition and the body temperature Tc that we want to obtain. Thus, we observe that the higher the inhalation temperature Ti, the greater the proportion of helium must be to maintain the body temperature Tc in a thermically neutral temperature range between 36° C. and 38° C.

More specifically, the distance H22-X22 corresponds to the difference between the body temperature of a rat breathing an oxygen-helium mixture and a body temperature of a rat breathing an oxygen-xenon mixture, with a same inhalation temperature of 22° C. The distance X22-T37 corresponds to the difference between the body temperature of a rat breathing an oxygen-xenon mixture for an inhalation temperature of 22° C., and a target body temperature of 37° C. Likewise, for an inhalation temperature of 22° C., the distances X22-T36, X22-T37 and X22-T38 correspond to the difference between the rat's body temperature breathing the oxygen-xenon mixture and the target body temperatures of 36° C. to 38° C.

Considering the functions represented by regression lines C1, C2, the proportions of the gaseous mixture to respect to obtain a hypothermic mixture were determined based on the calculation described below.

The curve C1 represents the function y=0.526x+20.748 and the curve C2 represents the function y=0.3877x+30.075. As an example, we look at a case with a desired body temperature of 37° C. with a room temperature of 22° C. and an oxygen rate of 22%, meaning an inert gas rate of 78%.

The first step is to calculate body temperatures: for an inhalation temperature substantially equal to 22° C., when a 22% O2-78% He mixture is breathed, we get a body temperature of 32.32° C. by using the representative function of the curve C1, and when a 22% O2-78% Xe mixture is breathed, we get a body temperature of 38.60° C. by using the representative function of the curve C2.

In the second step we find the difference, for the inhalation temperature of 22° C., between the body temperatures obtained through the calculations of the first step, which then is used as a reference value of the content calculations of each of the mixture's compounds: a first difference D1 is calculated between the body temperature obtained with a 22% O2-78% Xe mixture and the body temperature obtained with a 22% O2-78% He mixture, and in the case described, of an inhalation temperature equal to 22° C., here we have a value of 6.28.

In the third step, we calculate the content of one of the gases to be determined to get a body temperature of 37° C. for an inhalation temperature of 22° C. In the case described, we randomly chose to determine the content of helium, though we could have chosen to first determine the xenon content. A second difference D2 is calculated between the body temperature obtained with a 22% O2-78% Xe mixture and the desired body temperature for this inhalation temperature of 22° C., and here we have a value of 1.6.

This relationship between the values calculated in the second and third steps is used to cross calculate the product type to determine the content of helium out of the 78% of inert gases on top of oxygen, the gaseous composition to prepare to obtain a body temperature of 37° C.: In the case described, here we have a content equal to 20% (1.6×78/6.28) %. We subtract the xenon content (78−20=58) and, in this case, the composition will be composed of 58% helium, 22% oxygen and 20% xenon.

According to this example and the reading of table 2, for an inhalation temperature Ti of 26° C., a 22% oxygen proportion and a desired body temperature of 37° C., the composition contains 43% helium and 35% xenon.

We also observe that in all cases the composition contains between 5 and 71% helium. More specifically, when the oxygen content falls between 21 and 30%, the composition contains at least 7% helium and at most 71% xenon. According to this invention, we aim for a gaseous composition, which on one hand makes to possible to reach the target thermal properties, meaning the thermal properties obtained using a mixture of n=thermally inert neutral gases (the appropriate proportions to obtain this composition can be read in the table). The present invention also aims for a composition that can be used on subjects without risking an undesired anesthetic effect, meaning by limiting the xenon influx to a maximum of 50%. The resulting composition can substantially contain 21 to 30% oxygen, 11 to 64% helium, and 13 to 45% xenon. Preferably, the said composition contains 22% oxygen, 43% helium, and 35% xenon.

In the same way as described above, the graph in FIG. 2 represents experimental data Pi of the body temperature obtained in the rat, based on the helium (curve C1) or argon (curve C3) inhalation temperature, from which the proportions of the different gases in a helium-argon-oxygen mixture were calculated (table 3). As an example, reference points A27 and H27 used in this case were taken at an inhalation temperature Ti of 27° C., and the distances with the target body temperatures T36, T37 and T38 are therefore representative of the proportions of the inert gas mixture for this inhalation temperature of 27° C.

A comparison between graphs of FIGS. 1 and 2 highlight that the curve C3 has a lower slope than the curve C2. Indeed, the curve C2 represents the following function y=0.3877x+30.075 while the curve C3 represents the function y=0.2328x+32.334, argon representing lower hyperthermic properties than xenon. Thus, the proportions of inert gases in the inhalation gas composition, according to the invention, varies based on the quality of the first compound used in this composition, chosen from argon or xenon.

When reading table 3, we see that in any case, the composition includes at the most 67% argon and at least 8% helium. More specifically, when the oxygen content is between 21 and 30%, the composition contains at least 67% helium and at most 11% xenon. In addition, for inhalation temperatures Ti between 19° C. and 23° C., the composition contains 21 to 30% oxygen, 3 to 28% helium, and 46 to 76% argon.

Finally, these proportions make it possible to ensure that the mixture of inert gases is hypothermic. When the gaseous composition is inhaled at a given temperature Ti, it makes it possible to maintain the body temperature Tc of the inhaling subject within normal body temperature ranging from 36° C. to 38° C.

Means for inhaling such a composition include, but are not limited to, a human-machine interface, like a respiratory fan, a facial mask, respiratory goggles or any other kind of interface.

Furthermore, for reasons of security, and particular to avoid that one or several inert gases are inhaled, this kind of composition is preferably packaged into a single container with the three compounds, namely xenon or argon, helium and oxygen, in the pre-set proportions under a pressure between 10 and 300 bars. The container is 0.1 L to 50 L in volume. This packaging in a single bottle is called “ready-for-use.” In order to ensure a proportion of at least 21% oxygen in the composition and to always get a gaseous composition that can be inhaled, taking into account a 1% uncertainty between the different production steps, the packaging and administration of the gaseous composition, and in order to avoid hypoxia in the subject to whom the mixture is administered, the proportion of oxygen in this kind of packaging is always at least 22%.

### ANNEXES

TABLE 1

| Chemical | | | | |
|---|---|---|---|---|
| element | Nitrogen (N) | Xenon (Xe) | Argon (Ar) | Helium (He) |
| Molar mass | 28.013 | 131.29 | 39.948 | 4.003 |
| (mg/mol) | | | | |
| Thermal | 24.001 | 5.107 | 16.483 | 146.20 |
| conductivity | | | | |
| (mW/m · K) | | | | |

TABLE 2

| | % O2 = 21 | | | | | | % O2 = 22 | | | | | | % O2 = 23 | | | | | |
|---|---|---|---|---|---|---|---|---|---|---|---|---|---|---|---|---|---|---|
| | TC = 36° C. | | TC = 37° C. | | TC = 38° C. | | TC = 36° C. | | TC = 37° C. | | TC = 38° C. | | TC = 36° C. | | TC = 37° C. | | TC = 38° C. | |
| Ti | % He | % Xe | % He | % Xe | % He | % Xe | % He | % Xe | % He | % Xe | % He | % Xe | % He | % Xe | % He | % Xe | % He | % Xe |
| 16 | 3 | 76 | | | | | 3 | 75 | | | | | 3 | 74 | | | | |
| 17 | 8 | 71 | | | | | 7 | 71 | | | | | 7 | 70 | | | | |
| 18 | 12 | 67 | 1 | 7 | | | 12 | 66 | 1 | 77 | | | 12 | 65 | 1 | 76 | | |
| 19 | 17 | 62 | 5 | 74 | | | 17 | 61 | 5 | 73 | | | 17 | 60 | 5 | 72 | | |
| 20 | 22 | 57 | 10 | 69 | | | 22 | 56 | 10 | 68 | | | 21 | 36 | 10 | 67 | | |
| 21 | 27 | 52 | 15 | 64 | 3 | 76 | 27 | 51 | 15 | 63 | 3 | 75 | 27 | 50 | 15 | 62 | 3 | 74 |
| 22 | 33 | 46 | 20 | 59 | 8 | 71 | 32 | 46 | 20 | 58 | 8 | 70 | 32 | 45 | 20 | 57 | 7 | 70 |
| 23 | 38 | 41 | 26 | 53 | 13 | 66 | 38 | 40 | 25 | 53 | 13 | 65 | 37 | 40 | 25 | 52 | 12 | 65 |
| 24 | 44 | 35 | 31 | 48 | 18 | 61 | 44 | 34 | 31 | 47 | 18 | 60 | 43 | 34 | 31 | 46 | 18 | 59 |
| 25 | 51 | 28 | 37 | 42 | 24 | 55 | 50 | 28 | 37 | 41 | 23 | 55 | 49 | 28 | 36 | 41 | 23 | 54 |
| 26 | 57 | 22 | 43 | 36 | 30 | 49 | 57 | 21 | 43 | 35 | 29 | 49 | 56 | 21 | 42 | 35 | 29 | 48 |
| 27 | 64 | 15 | 50 | 29 | 36 | 43 | 63 | 15 | 49 | 29 | 35 | 43 | 63 | 14 | 49 | 28 | 35 | 42 |
| 28 | 71 | 8 | 57 | 22 | 42 | 37 | 71 | 7 | 56 | 22 | 42 | 36 | 70 | 7 | 55 | 22 | 41 | 36 |
| | % O2 = 24 | | | | | | % O2 = 25 | | | | | | % O2 = 26 | | | | | |
| | TC = 36° C. | | TC = 37° C. | | TC = 38° C. | | TC = 36° C. | | TC = 37° C. | | TC = 38° C. | | TC = 36° C. | | TC = 37° C. | | TC = 38° C. | |
| Ti | % He | % Xe | % He | % Xe | % He | % Xe | % He | % Xe | % He | % Xe | % He | % Xe | % He | % Xe | % He | % Xe | % He | % Xe |
| 16 | 3 | 73 | | | | | 3 | 72 | | | | | 3 | 71 | | | | |
| 17 | 7 | 69 | | | | | 7 | 68 | | | | | 7 | 67 | | | | |
| 18 | 12 | 64 | 1 | 75 | | | 12 | 63 | 1 | 74 | | | 11 | 63 | 1 | 75 | | |
| 19 | 16 | 60 | 5 | 71 | | | 16 | 59 | 5 | 70 | | | 16 | 58 | 5 | 69 | | |
| 20 | 21 | 55 | 10 | 66 | | | 21 | 54 | 9 | 66 | | | 21 | 53 | 9 | 65 | | |
| 21 | 26 | 50 | 14 | 62 | 3 | 73 | 26 | 49 | 14 | 61 | 3 | 72 | 26 | 48 | 14 | 60 | 2 | 72 |
| 22 | 31 | 45 | 19 | 57 | 7 | 69 | 31 | 44 | 19 | 56 | 7 | 68 | 31 | 43 | 19 | 55 | 7 | 67 |
| 23 | 37 | 39 | 25 | 51 | 12 | 64 | 37 | 38 | 24 | 51 | 12 | 63 | 36 | 38 | 24 | 50 | 12 | 62 |
| 24 | 43 | 33 | 30 | 46 | 17 | 59 | 42 | 33 | 30 | 45 | 17 | 58 | 42 | 32 | 29 | 45 | 17 | 57 |
| 25 | 49 | 27 | 36 | 40 | 23 | 53 | 48 | 27 | 35 | 40 | 23 | 52 | 47 | 27 | 35 | 39 | 22 | 52 |
| 26 | 55 | 21 | 42 | 34 | 29 | 47 | 54 | 21 | 41 | 34 | 28 | 47 | 54 | 20 | 41 | 33 | 28 | 46 |
| 27 | 62 | 14 | 48 | 28 | 35 | 41 | 61 | 14 | 48 | 27 | 34 | 41 | 60 | 14 | 47 | 27 | 34 | 40 |
| 28 | 69 | 7 | 55 | 21 | 41 | 35 | 68 | 7 | 54 | 21 | 40 | 35 | 67 | 7 | 53 | 21 | 40 | 34 |
| | % O2 = 27 | | | | | | % O2 = 28 | | | | | | % O2 = 29 | | | | | |
| | TC = 36° C. | | TC = 37° C. | | TC = 38° C. | | TC = 36° C. | | TC = 37° C. | | TC = 38° C. | | TC = 36° C. | | TC = 37° C. | | TC = 38° C. | |
| Ti | % He | % Xe | % He | % Xe | % He | % Xe | % He | % Xe | % He | % Xe | % He | % Xe | % He | % Xe | % He | % Xe | % He | % Xe |
| 16 | 3 | 70 | | | | | 3 | 69 | | | | | 3 | 68 | | | | |
| 17 | 7 | 66 | | | | | 7 | 65 | | | | | 7 | 64 | | | | |
| 18 | 11 | 62 | 1 | 72 | | | 11 | 61 | 1 | 71 | | | 11 | 60 | 1 | 70 | | |
| 19 | 16 | 57 | 5 | 68 | | | 15 | 57 | 5 | 67 | | | 15 | 56 | 5 | 66 | | |
| 20 | 20 | 53 | 9 | 64 | | | 20 | 52 | 9 | 73 | | | 20 | 51 | 9 | 62 | | |
| 21 | 25 | 48 | 14 | 59 | 2 | 71 | 25 | 47 | 14 | 58 | 2 | 70 | 25 | 46 | 13 | 58 | 2 | 69 |
| 22 | 30 | 43 | 19 | 54 | 7 | 66 | 30 | 42 | 18 | 54 | 7 | 65 | 29 | 42 | 18 | 53 | 7 | 64 |
| 23 | 36 | 37 | 24 | 49 | 12 | 61 | 35 | 37 | 23 | 49 | 12 | 60 | 35 | 36 | 23 | 48 | 11 | 60 |
| 24 | 41 | 32 | 29 | 44 | 17 | 56 | 41 | 31 | 29 | 43 | 17 | 55 | 40 | 31 | 28 | 43 | 16 | 55 |
| 25 | 47 | 26 | 34 | 39 | 22 | 51 | 46 | 26 | 34 | 38 | 22 | 50 | 46 | 25 | 33 | 38 | 21 | 50 |
| 26 | 53 | 20 | 40 | 33 | 27 | 46 | 52 | 20 | 40 | 32 | 27 | 45 | 51 | 20 | 39 | 32 | 27 | 44 |
| 27 | 59 | 14 | 46 | 27 | 33 | 40 | 58 | 14 | 46 | 26 | 33 | 39 | 58 | 13 | 45 | 26 | 32 | 39 |
| 28 | 66 | 7 | 53 | 20 | 39 | 34 | 65 | 7 | 52 | 20 | 39 | 33 | 64 | 7 | 51 | 20 | 38 | 33 |
| | % O2 = 30 | | | | | | % O2 = 35 | | | | | | % O2 = 40 | | | | | |
| | TC = 36° C. | | TC = 37° C. | | TC = 38° C. | | TC = 36° C. | | TC = 37° C. | | TC = 38° C. | | TC = 36° C. | | TC = 37° C. | | TC = 38° C. | |
| Ti | % He | % Xe | % He | % Xe | % He | % Xe | % He | % Xe | % He | % Xe | % He | % Xe | % He | % Xe | % He | % Xe | % He | % Xe |
| 16 | 3 | 67 | | | | | 3 | 62 | | | | | 2 | 58 | | | | |
| 17 | 7 | 63 | | | | | 6 | 59 | | | | | 6 | 54 | | | | |
| 18 | 11 | 59 | 1 | 69 | | | 10 | 55 | 1 | 64 | | | 9 | 51 | | | | |
| 19 | 15 | 55 | 5 | 65 | | | 14 | 51 | 4 | 61 | | | 13 | 47 | 4 | 56 | | |
| 20 | 20 | 50 | 9 | 61 | | | 18 | 47 | 8 | 57 | | | 17 | 43 | 8 | 52 | | |
| 21 | 24 | 46 | 13 | 57 | 2 | 68 | 22 | 43 | 12 | 53 | 2 | 63 | 21 | 39 | 11 | 49 | 2 | 58 |
| 22 | 29 | 41 | 18 | 52 | 7 | 63 | 27 | 38 | 17 | 48 | 6 | 59 | 25 | 35 | 15 | 45 | 6 | 54 |
| 23 | 34 | 36 | 23 | 47 | 11 | 59 | 32 | 33 | 21 | 44 | 10 | 55 | 29 | 31 | 19 | 41 | 10 | 50 |
| 24 | 39 | 31 | 28 | 42 | 16 | 54 | 37 | 28 | 26 | 39 | 15 | 50 | 34 | 26 | 24 | 36 | 14 | 46 |
| 25 | 45 | 25 | 33 | 37 | 21 | 49 | 42 | 23 | 31 | 34 | 20 | 45 | 39 | 21 | 28 | 32 | 18 | 42 |
| 26 | 51 | 19 | 39 | 31 | 26 | 44 | 47 | 18 | 36 | 29 | 24 | 41 | 44 | 16 | 33 | 27 | 23 | 37 |
| 27 | 57 | 13 | 44 | 26 | 32 | 38 | 53 | 12 | 41 | 24 | 30 | 35 | 49 | 11 | 38 | 22 | 27 | 33 |
| 28 | 63 | 7 | 50 | 20 | 38 | 32 | 59 | 6 | 47 | 18 | 35 | 30 | 54 | 6 | 43 | 17 | 32 | 28 |

| | % O2 = 45 | | | | | | % O2 = 50 | | | | | |
|---|---|---|---|---|---|---|---|---|---|---|---|---|
| | TC = 36° C. | | TC = 37° C. | | TC = 38° C. | | TC = 36° C. | | TC = 37° C. | | TC = 38° C. | |
| Ti | % He | % Xe | % He | % Xe | % He | % Xe | % He | % Xe | % He | % Xe | % He | % Xe |
| 16 | 2 | 53 | | | | | 2 | 48 | | | | |
| 17 | 5 | 50 | | | | | 5 | 45 | | | | |
| 18 | 8 | 47 | | | | | 8 | 42 | | | | |
| 19 | 12 | 43 | 4 | 51 | | | 11 | 39 | 3 | 47 | | |
| 20 | 15 | 40 | 7 | 48 | | | 14 | 36 | 6 | 44 | | |
| 21 | 19 | 36 | 10 | 45 | 2 | 53 | 17 | 33 | 9 | 41 | 2 | 48 |
| 22 | 23 | 32 | 14 | 41 | 5 | 50 | 21 | 29 | 13 | 37 | 5 | 45⁵ |
| 23 | 27 | 28 | 18 | 37 | 9 | 46 | 24 | 26 | 16 | 34 | 8 | 42 |
| 24 | 31 | 24 | 22 | 33 | 13 | 42 | 28 | 22 | 20 | 30 | 11 | 39 |
| 25 | 35 | 20 | 26 | 29 | 17 | 38 | 32 | 18 | 24 | 26 | 15 | 35 |
| 26 | 40 | 15 | 30 | 25 | 21 | 34 | 36 | 14 | 28 | 22 | 19 | 31 |
| 27 | 45 | 10 | 35 | 20 | 25 | 30 | 41 | 9 | 32 | 18 | 23 | 27 |
| 28 | 50 | 5 | 40 | 15 | 30 | 25 | 45 | 5 | 36 | 14 | 27 | 23 |

TABLE 3

| | % O2 = 21 | | | | | | % O2 = 22 | | | | | | % O2 = 23 | | | | | |
|---|---|---|---|---|---|---|---|---|---|---|---|---|---|---|---|---|---|---|
| | TC = 36° C. | | TC = 37° C. | | TC = 38° C. | | TC = 36° C. | | TC = 37° C. | | TC = 38° C. | | TC = 36° C. | | TC = 37° C. | | TC = 38° C. | |
| Ti | % He | % Ar | % He | % Ar | % He | % Ar | % He | % Ar | % He | % Ar | % He | % Ar | % He | % Ar | % He | % Ar | % He | % Ar |
| 16 | 1 | 78 | | | | | 1 | 77 | | | | | 1 | 76 | | | | |
| 17 | 3 | 76 | | | | | 3 | 75 | | | | | 3 | 74 | | | | |
| 18 | 7 | 72 | | | | | 6 | 72 | | | | | 6 | 71 | | | | |
| 19 | 10 | 69 | | | | | 10 | 68 | | | | | 10 | 67 | | | | |
| 20 | 14 | 65 | | | | | 13 | 65 | | | | | 13 | 64 | | | | |
| 21 | 18 | 61 | 3 | 76 | | | 18 | 60 | 3 | 75 | | | 17 | 60 | 3 | 74 | | |
| 22 | 22 | 57 | 7 | 72 | | | 22 | 56 | 7 | 71 | | | 22 | 55 | 7 | 70 | | |
| 23 | 28 | 51 | 11 | 68 | | | 27 | 51 | 11 | 67 | | | 27 | 50 | 11 | 66 | | |
| 24 | 33 | 46 | 16 | 63 | | | 33 | 45 | 16 | 62 | | | 33 | 44 | 16 | 61 | | |
| 25 | 40 | 39 | 21 | 58 | 3 | 76 | 39 | 39 | 21 | 57 | 3 | 75 | 39 | 38 | 21 | 56 | 3 | 74 |
| 26 | 48 | 31 | 28 | 51 | 8 | 71 | 47 | 31 | 27 | 51 | 8 | 70 | 46 | 31 | 27 | 58 | 8 | 69 |
| 27 | 56 | 23 | 35 | 44 | 13 | 66 | 56 | 22 | 34 | 44 | 13 | 65 | 55 | 22 | 34 | 43 | 13 | 64 |
| 28 | 67 | 12 | 43 | 36 | 20 | 59 | 66 | 12 | 43 | 35 | 20 | 58 | 65 | 12 | 42 | 35 | 19 | 58 |
| | % O2 = 24 | | | | | | % O2 = 25 | | | | | | % O2 = 26 | | | | | |
| | TC = 36° C. | | TC = 37° C. | | TC = 38° C. | | TC = 36° C. | | TC = 37° C. | | TC = 38° C. | | TC = 36° C. | | TC = 37° C. | | TC = 38° C. | |
| Ti | % He | % Ar | % He | % Ar | % He | % Ar | % He | % Ar | % He | % Ar | % He | % Ar | % He | % Ar | % He | % Ar | % He | % Ar |
| 16 | 1 | 75 | | | | | 1 | 74 | | | | | 1 | 73 | | | | |
| 17 | 3 | 73 | | | | | 3 | 72 | | | | | 3 | 71 | | | | |
| 18 | 6 | 70 | | | | | 6 | 69 | | | | | 6 | 68 | | | | |
| 19 | 10 | 66 | | | | | 9 | 66 | | | | | 9 | 65 | | | | |
| 20 | 13 | 63 | | | | | 13 | 62 | | | | | 13 | 61 | | | | |
| 21 | 17 | 59 | 3 | 73 | | | 17 | 58 | 3 | 72 | | | 17 | 57 | 3 | 71 | | |
| 22 | 22 | 54 | 7 | 69 | | | 21 | 54 | 7 | 68 | | | 21 | 53 | 7 | 67 | | |
| 23 | 26 | 50 | 11 | 65 | | | 26 | 49 | 11 | 64 | | | 26 | 48 | 11 | 63 | | |
| 24 | 32 | 44 | 15 | 61 | | | 32 | 43 | 15 | 60 | | | 31 | 43 | 15 | 59 | | |
| 25 | 38 | 38 | 21 | 55 | 3 | 73 | 38 | 37 | 20 | 55 | 3 | 72 | 37 | 37 | 20 | 54 | 3 | 71 |
| 26 | 46 | 30 | 27 | 49 | 7 | 69 | 45 | 30 | 26 | 49 | 7 | 68 | 45 | 29 | 26 | 48 | 7 | 67 |
| 27 | 54 | 22 | 34 | 42 | 13 | 63 | 54 | 21 | 33 | 42 | 13 | 62 | 53 | 21 | 33 | 41 | 12 | 62 |
| 28 | 64 | 12 | 42 | 34 | 19 | 57 | 63 | 12 | 41 | 34 | 19 | 56 | 63 | 11 | 41 | 33 | 19 | 55 |
| | % O2 = 27 | | | | | | % O2 = 28 | | | | | | % O2 = 29 | | | | | |
| | TC = 36° C. | | TC = 37° C. | | TC = 38° C. | | TC = 36° C. | | TC = 37° C. | | TC = 38° C. | | TC = 36° C. | | TC = 37° C. | | TC = 38° C. | |
| Ti | % He | % Ar | % He | % Ar | % He | % Ar | % He | % Ar | % He | % Ar | % He | % Ar | % He | % Ar | % He | % Ar | % He | % Ar |
| 16 | 1 | 72 | | | | | 1 | 71 | | | | | 1 | 70 | | | | |
| 17 | 3 | 70 | | | | | 3 | 69 | | | | | 3 | 68 | | | | |
| 18 | 6 | 67 | | | | | 6 | 66 | | | | | 6 | 65 | | | | |
| 19 | 9 | 64 | | | | | 9 | 63 | | | | | 9 | 62 | | | | |
| 20 | 13 | 60 | | | | | 12 | 60 | | | | | 12 | 59 | | | | |
| 21 | 16 | 57 | 3 | 70 | | | 16 | 56 | 3 | 69 | | | 16 | 55 | 3 | 68 | | |
| 22 | 21 | 52 | 6 | 67 | | | 20 | 52 | 6 | 66 | | | 20 | 51 | 6 | 65 | | |
| 23 | 25 | 48 | 10 | 63 | | | 25 | 47 | 10 | 62 | | | 25 | 46 | 10 | 61 | | |
| 24 | 31 | 42 | 15 | 58 | | | 30 | 42 | 15 | 57 | | | 30 | 41 | 14 | 57 | | |
| 25 | 37 | 36 | 20 | 53 | 3 | 70 | 36 | 36 | 20 | 52 | 3 | 69 | 36 | 35 | 19 | 52 | 3 | 68 |
| 26 | 44 | 29 | 26 | 47 | 7 | 66 | 43 | 29 | 25 | 47 | 7 | 65 | 43 | 28 | 25 | 46 | 7 | 64 |
| 27 | 52 | 21 | 32 | 41 | 12 | 61 | 51 | 21 | 32 | 40 | 12 | 60 | 51 | 20 | 31 | 40 | 12 | 59 |
| 28 | 62 | 11 | 40 | 33 | 18 | 55 | 61 | 11 | 40 | 32 | 18 | 54 | 60 | 11 | 39 | 32 | 18 | 53 |
| | % O2 = 30 | | | | | | % O2 = 35 | | | | | | % O2 = 40 | | | | | |
| | TC = 36° C. | | TC = 37° C. | | TC = 38° C. | | TC = 36° C. | | TC = 37° C. | | TC = 38° C. | | TC = 36° C. | | TC = 37° C. | | TC = 38° C. | |
| Ti | % He | % Ar | % He | % Ar | % He | % Ar | % He | % Ar | % He | % Ar | % He | % Ar | % He | % Ar | % He | % Ar | % He | % Ar |
| 16 | 1 | 69 | | | | | 1 | 64 | | | | | 1 | 59 | | | | |
| 17 | 3 | 67 | | | | | 3 | 62 | | | | | 3 | 57 | | | | |
| 18 | 6 | 64 | | | | | 5 | 60 | | | | | 5 | 55 | | | | |
| 19 | 9 | 61 | | | | | 8 | 57 | | | | | 8 | 52 | | | | |
| 20 | 12 | 58 | | | | | 11 | 54 | | | | | 10 | 50 | | | | |
| 21 | 16 | 54 | 3 | 67 | | | 15 | 50 | 3 | 62 | | | 14 | 46 | 2 | 58 | | |
| 22 | 20 | 50 | 6 | 64 | | | 18 | 47 | 6 | 59 | | | 17 | 43 | 5 | 55 | | |
| 23 | 24 | 46 | 10 | 60 | | | 23 | 42 | 9 | 56 | | | 21 | 39 | 9 | 51 | | |
| 24 | 30 | 40 | 14 | 56 | | | 27 | 38 | 13 | 52 | | | 25 | 35 | 12 | 48 | | |
| 25 | 35 | 35 | 19 | 51 | 3 | 67 | 33 | 32 | 18 | 47 | 2 | 63 | 30 | 30 | 16 | 44 | 2 | 58 |
| 26 | 42 | 28 | 24 | 46 | 7 | 63 | 39 | 26 | 23 | 42 | 6 | 59 | 36 | 24 | 21 | 39 | 6 | 54 |
| 27 | 50 | 20 | 31 | 39 | 12 | 58 | 46 | 19 | 29 | 36 | 11 | 54 | 43 | 17 | 26 | 34 | 10 | 50 |
| 28 | 59 | 11 | 38 | 32 | 18 | 52 | 55 | 10 | 36 | 29 | 16 | 49 | 51 | 9 | 33 | 27 | 15 | 45 |

| | % O2 = 45 | | | | | | % O2 = 50 | | | | | |
|---|---|---|---|---|---|---|---|---|---|---|---|---|
| | TC = 36° C. | | TC = 37° C. | | TC = 38° C. | | TC = 36° C. | | TC = 37° C. | | TC = 38° C. | |
| Ti | % He | % Ar | % He | % Ar | % He | % Ar | % He | % Ar | % He | % Ar | % He | % Ar |
| 16 | | | | | | | | | | | | |
| 17 | 2 | 53 | | | | | 2 | 48 | | | | |
| 18 | 5 | 50 | | | | | 4 | 46 | | | | |
| 19 | 7 | 48 | | | | | 6 | 44 | | | | |
| 20 | 10 | 45 | | | | | 9 | 41 | | | | |
| 21 | 12 | 43 | 2 | 53 | | | 11 | 39 | 2 | 48 | | |
| 22 | 16 | 39 | 5 | 50 | | | 14 | 36 | 4 | 46 | | |
| 23 | 19 | 36 | 8 | 47 | | | 17 | 33 | 7 | 43 | | |
| 24 | 23 | 32 | 11 | 44 | | | 21 | 29 | 10 | 40 | | |
| 25 | 28 | 27 | 15 | 40 | 2 | 53 | 25 | 25 | 14 | 36 | 2 | 48 |
| 26 | 33 | 22 | 19 | 36 | 5 | 50 | 30 | 20 | 17 | 33 | 5 | 45 |
| 27 | 39 | 16 | 24 | 31 | 9 | 46 | 36 | 14 | 22 | 28 | 8 | 42 |
| 28 | 46 | 9 | 30 | 25 | 14 | 14 | 42 | 8 | 27 | 23 | 13 | 37 |

1-17. (canceled)

18. A method to administer to a patient a thermally neutral inhalation gas composition, comprising:
selecting a gas composition with 23% to 27% oxygen, 43% to 46% helium and 28 to 32% xenon; and
administering the gas composition to the patient either at an inhalation temperature of 24° C. or 25° C. in order to get a body temperature of the patient of 36° C., or at an inhalation temperature of 26° C. or 27° C. in order to get a body temperature of the patient of 37° C.

19. The method of claim 18, wherein the selected gas composition comprises 25% oxygen, 45% helium and 30% xenon.

20. The method of claim 18, wherein the step of administering the gas composition is performed via human-machine interface.

21. The method of claim 19, wherein the human-machine interface is a respiratory fan, a facial mask, or respiratory goggles.

22. The method of claim 18, further comprising a step of packaging the selected gas composition into a single container, wherein the packaging step is performed between the selecting step and the administering step.

23. The method according to claim 22, wherein oxygen, helium and xenon are packaged in the single container under a pressure between 10 and 300 bars.

24. A method to administer to a patient a thermally neutral inhalation gas composition, comprising:
selecting a gas composition with 24% to 26% oxygen, 43% to 47% helium and 28% to 32% xenon; and
administering the gas composition to the patient at an inhalation temperature of 24° C. or 25° C. in order to get a body temperature of the patient of 36° C., or at an inhalation temperature of 26° C. or 27° C. in order to get a body temperature of the patient of 37° C.

25. The method of claim 24, wherein the step of administering the gas composition is performed via human-machine interface.

26. The method of claim 25, wherein the human-machine interface is a respiratory fan, a facial mask, or respiratory goggles.

27. The method of claim 24, further comprising a step of packaging the selected gas composition into a single container, wherein the packaging step is performed between the selecting step and the administering step.

28. The method of claim 27, wherein oxygen, helium and xenon are packaged in the single container under a pressure between 10 and 300 bars.

29. A method to administer to a patient a thermally neutral inhalation gas composition, comprising:
selecting a gas composition with 21% to 30% oxygen, 37% to 45% helium and 28% to 37% xenon; and
administering the gas composition to the patient at an inhalation temperature between 22° C. and 25° C. in order to get a body temperature of the patient of 36° C., or at an inhalation temperature between 25° C. or 28° C. in order to get a body temperature of the patient of 37° C.

30. The method of claim 29, wherein the step of administering the gas composition is performed via human-machine interface.

31. The method of claim 30, wherein the human-machine interface is a respiratory fan, a facial mask, or respiratory goggles.

32. The method of claim 29, further comprising a step of packaging the selected gas composition into a single container, wherein the packaging step is performed between the selecting step and the administering step.

33. The method of claim 32, wherein oxygen, helium and xenon are packaged in the single container under a pressure between 10 and 300 bars.
